SEQUENCE LISTING (86 sequences)

SEQ ID NO: 1 - protein, 5 aa, synthetic construct
NHDMG

SEQ ID NO: 2 - protein, 10 aa, synthetic construct
PKPMDTEFDY

SEQ ID NO: 3 - protein, 11 aa, synthetic construct
EPKPMDTEFDY

SEQ ID NO: 4 - protein, 119 aa, synthetic construct
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSS

SEQ ID NO: 5 - protein, 30 aa, synthetic construct
EVQLLESGGGLVQPGGSLRLSCAASGFTFR

SEQ ID NO: 6 - protein, 14 aa, synthetic construct
WVRQAPGKGLEWVS

SEQ ID NO: 7 - protein, 32 aa, synthetic construct
RFTISRDNSKNTLYLQMNSLRAEDTAVYYCAE

SEQ ID NO: 8 - protein, 11 aa, synthetic construct
RSPGTLVTVSS

SEQ ID NO: 9 - protein, 11 aa, synthetic construct
RASQYIYDYLN

SEQ ID NO: 10 - protein, 7 aa, synthetic construct
AASYLQS

SEQ ID NO: 11 - protein, 8 aa, synthetic construct
QQYHHPST

SEQ ID NO: 12 - protein, 107 aa, synthetic construct
DIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKR

SEQ ID NO: 13 - protein, 23 aa, synthetic construct
DIQMTQSPSSLSASVGDRVTITC

SEQ ID NO: 14 - protein, 15 aa, synthetic construct
WYQQKPGKAPKLLIY

SEQ ID NO: 15 - protein, 32 aa, synthetic construct
GVPSRFSGSGSGTDFTLTISSLQPEDFATYYC

SEQ ID NO: 16 - protein, 11 aa, synthetic construct
FGQGTKVEIKR

SEQ ID NO: 17 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 18 - protein, 16 aa, synthetic construct
GHNYTTNILPGLNITC

SEQ ID NO: 19 - protein, 15 aa, synthetic construct
KYYKENNVEKRTLIK

SEQ ID NO: 20 - protein, 25 aa, synthetic construct
GHNYTTRNILPGAGAKYYKENNVEK

SEQ ID NO: 21 - protein, 11 aa, synthetic construct
RASQSISSYLN

SEQ ID NO: 22 - protein, 7 aa, synthetic construct
AASSLQS

SEQ ID NO: 23 - protein, 9 aa, synthetic construct
QQFDYMPLT

SEQ ID NO: 24 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQFDYMPLTFGQGTKVEIKR

SEQ ID NO: 25 - protein, 23 aa, synthetic construct
DIQMTQSPSSLSASVGDRVTITC

SEQ ID NO: 26 - protein, 15 aa, synthetic construct
WYQQKPGKAPKLLIY

SEQ ID NO: 27 - protein, 32 aa, synthetic construct; identical to SEQ ID NO: 15

SEQ ID NO: 28 - protein, 11 aa, synthetic construct
FGQGTKVEIKR

SEQ ID NO: 29 - protein, 7 aa, synthetic construct
GFTFRNH

SEQ ID NO: 30 - protein, 6 aa, synthetic construct
SGSGGS

SEQ ID NO: 31 - protein, 10 aa, synthetic construct
PKPMDTEFDY

SEQ ID NO: 32 - protein, 25 aa, synthetic construct
EVQLLESGGGLVQPGGSLRLSCAAS

SEQ ID NO: 33 - protein, 19 aa, synthetic construct
DMGWVRQAPGKGLEWVSAI

SEQ ID NO: 34 - protein, 41 aa, synthetic construct
TYYANSVKGRFTISRDNSKNTLYLQMNSLRAEDTAVYYCAE

SEQ ID NO: 35 - protein, 11 aa, synthetic construct
RSPGTLVTVSS

SEQ ID NO: 36 - protein, 8 aa, synthetic construct
GFTFRNHD

SEQ ID NO: 37 - protein, 8 aa, synthetic construct
ISGSGGST

SEQ ID NO: 38 - protein, 12 aa, synthetic construct
AEPKPMDTEFDY

SEQ ID NO: 39 - protein, 25 aa, synthetic construct
EVQLLESGGGLVQPGGSLRLSCAAS

SEQ ID NO: 40 - protein, 17 aa, synthetic construct
MGWVRQAPGKGLEWVSA

SEQ ID NO: 41 - protein, 38 aa, synthetic construct
YYANSVKGRFTISRDNSKNTLYLQMNSLRAEDTAVYYC

SEQ ID NO: 42 - protein, 11 aa, synthetic construct
RSPGTLVTVSS

SEQ ID NO: 43 - protein, 5 aa, synthetic construct
NHDMG

SEQ ID NO: 44 - protein, 17 aa, synthetic construct
AISGSGGSTYYANSVKG

SEQ ID NO: 45 - protein, 10 aa, synthetic construct
PKPMDTEFDY

SEQ ID NO: 46 - protein, 30 aa, synthetic construct; identical to SEQ ID NO: 5

SEQ ID NO: 47 - protein, 14 aa, synthetic construct
WVRQAPGKGLEWVS

SEQ ID NO: 48 - protein, 32 aa, synthetic construct; identical to SEQ ID NO: 7

SEQ ID NO: 49 - protein, 11 aa, synthetic construct
RSPGTLVTVSS

SEQ ID NO: 50 - protein, 11 aa, synthetic construct
RASQYIYDYLN

SEQ ID NO: 51 - protein, 7 aa, synthetic construct
AASYLQS

SEQ ID NO: 52 - protein, 8 aa, synthetic construct
QQYHHPST

SEQ ID NO: 53 - protein, 23 aa, synthetic construct
DIQMTQSPSSLSASVGDRVTITC

SEQ ID NO: 54 - protein, 15 aa, synthetic construct
WYQQKPGKAPKLLIY

SEQ ID NO: 55 - protein, 32 aa, synthetic construct; identical to SEQ ID NO: 15

SEQ ID NO: 56 - protein, 10 aa, synthetic construct
FGQGTKVEIK

SEQ ID NO: 57 - protein, 6 aa, synthetic construct
QYIYDY

SEQ ID NO: 58 - skipped

SEQ ID NO: 59 - protein, 8 aa, synthetic construct
QQYHHPST

SEQ ID NO: 60 - protein, 26 aa, synthetic construct [sequence omitted]

SEQ ID NO: 61 - protein, 17 aa, synthetic construct
LNWYQQKPGKAPKLLIY

SEQ ID NO: 62 - protein, 36 aa, synthetic construct
YLQSGVPSRFSGSGSGTDFTLTISSLQPEDFATYYC

SEQ ID NO: 63 - protein, 10 aa, synthetic construct
FGQGTKVEIK

SEQ ID NO: 64 - protein, 6 aa, synthetic construct
QYIYDY

SEQ ID NO: 65 - skipped

SEQ ID NO: 66 - protein, 8 aa, synthetic construct
QQYHHPST

SEQ ID NO: 67 - protein, 26 aa, synthetic construct [sequence omitted]

SEQ ID NO: 68 - protein, 17 aa, synthetic construct
LNWYQQKPGKAPKLLIY

SEQ ID NO: 69 - protein, 36 aa, synthetic construct; identical to SEQ ID NO: 62

SEQ ID NO: 70 - protein, 10 aa, synthetic construct
FGQGTKVEIK

SEQ ID NO: 71 - DNA, 15 bp, synthetic construct
  source 1..15: mol_type = other DNA
aaccatgatatgggc

SEQ ID NO: 72 - DNA, 51 bp, synthetic construct
  source 1..51: mol_type = other DNA
gcgattagcggcagcggcggcagcacctattatgcgaacagcgtgaaaggc

SEQ ID NO: 73 - DNA, 30 bp, synthetic construct
  source 1..30: mol_type = other DNA
ccgaaaccgatggataccgaatttgattat

SEQ ID NO: 74 - DNA, 357 bp, synthetic construct
  source 1..357: mol_type = other DNA
gaagtgcagctgctggaaagcggcggcggcctggtgcagccgggcggcagcctgcgcctgagctgcgcggcgagcggctttacctttcgcaaccatgatatgggctgggtgcgccaggcgccgggcaaaggcctggaatgggtgagcgcgattagcggcagcggcggcagcacctattatgcgaacagcgtgaaaggccgctttaccattagccgcgataacagcaaaaacaccctgtatctgcagatgaacagcctgcgcgcggaagataccgcggtgtattattgcgcggaaccgaaaccgatggataccgaatttgattatcgcagcccgggcaccctggtgaccgtgagcagc

SEQ ID NO: 75 - DNA, 90 bp, synthetic construct
  source 1..90: mol_type = other DNA
gaagtgcagctgctggaaagcggcggcggcctggtgcagccgggcggcagcctgcgcctgagctgcgcggcgagcggctttacctttcgc

SEQ ID NO: 76 - DNA, 42 bp, synthetic construct
  source 1..42: mol_type = other DNA
tgggtgcgccaggcgccgggcaaaggcctggaatgggtgagc

SEQ ID NO: 77 - DNA, 96 bp, synthetic construct
  source 1..96: mol_type = other DNA
cgctttaccattagccgcgataacagcaaaaacaccctgtatctgcagatgaacagcctgcgcgcggaagataccgcggtgtattattgcgcggaa

SEQ ID NO: 78 - DNA, 33 bp, synthetic construct
  source 1..33: mol_type = other DNA
cgcagcccgggcaccctggtgaccgtgagcagc

SEQ ID NO: 79 - DNA, 33 bp, synthetic construct
  source 1..33: mol_type = other DNA
cgcgcgagccagagcattagcagctatctgaac

SEQ ID NO: 80 - DNA, 21 bp, synthetic construct
  source 1..21: mol_type = other DNA
gcggcgagcagcctgcagagc

SEQ ID NO: 81 - DNA, 27 bp, synthetic construct
  source 1..27: mol_type = other DNA
cagcagtttgattatatgccgctgacc

SEQ ID NO: 82 - DNA, 324 bp, synthetic construct
  source 1..324: mol_type = other DNA
gatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagtttgattatatgccgctgacctttggccagggcaccaaagtggaaattaaacgc

SEQ ID NO: 83 - DNA, 69 bp, synthetic construct
  source 1..69: mol_type = other DNA
gatattcagatgacccagagcccgagcagcctgagcgcgagcgtgggcgatcgcgtgaccattacctgc

SEQ ID NO: 84 - DNA, 45 bp, synthetic construct
  source 1..45: mol_type = other DNA
tggtatcagcagaaaccgggcaaagcgccgaaactgctgatttat

SEQ ID NO: 85 - DNA, 96 bp, synthetic construct
  source 1..96: mol_type = other DNA
ggcgtgccgagccgctttagcggcagcggcagcggcaccgattttaccctgaccattagcagcctgcagccggaagattttgcgacctattattgc

SEQ ID NO: 86 - DNA, 33 bp, synthetic construct
  source 1..33: mol_type = other DNA
tttggccagggcaccaaagtggaaattaaacgc

(19) United States
(12) Patent Application Publication
(10) Pub. No.: US 2024/0376193 A1
(43) Pub. Date: Nov. 14, 2024

(54) NOVEL DYSFUNCTIONAL P2X7 BINDERS

(71) Applicants: Patrick Schlegel, North Ryde New South Wales (AU); Julian Alexander Barden, New Ryde, New South Wales (AU); Daniel Ulrich Christ, North Ryde, New South Wales (AU); Peter Robert Schofield, Carlton (AU)

(72) Inventors: Patrick Schlegel, North Ryde New South Wales (AU); Julian Alexander Barden, New Ryde, New South Wales (AU); Daniel Ulrich Christ, North Ryde, New South Wales (AU); Peter Robert Schofield, Carlton (AU)

(73) Assignee: BIOSCEPTRE (AUST) PTY LTD, New South Wales (AU)

(21) Appl. No.: 18/686,554

(22) PCT Filed: Sep. 1, 2022
(86) PCT No.: PCT/AU2022/051066

§ 371 Date: Feb. 26, 2024

(30) Foreign Application Priority Data
Sep. 1, 2021 (AU) 2021902822

(51) Int. Cl. C07K 16/28

(52) U.S. Cl. CPC C07K 16/28; C07K 2317/565; C07K 2317/567; C07K 2317/732; C07K 2317/92

(57) ABSTRACT

The present invention relates to novel antigen binding proteins for binding to dysfunctional P2X₇ receptor.

### FIELD OF THE INVENTION

The invention relates to antigen binding proteins and related fragments thereof for binding to dysfunctional P2X₇ receptor, to production of said antigen binding proteins and fragments and to use of said antibodies and fragments for detection and therapy of various conditions.

### RELATED APPLICATION

This application claims priority from Australian provisional application AU 2021902832, the entire contents of which are hereby incorporated by reference.

### BACKGROUND OF THE INVENTION

Purinergic (P2X) receptors are ATP-gated cation-selective channels. Each receptor is made up of three protein subunits or monomers. To date seven separate genes encoding P2X monomers have been identified: P2X₁, P2X₂, P2X₃, P2X₄, P2X₅, P2X₆, P2X₇.

P2X₇ receptors are of particular interest as the expression of these receptors is understood to be limited to cells having potential to undergo programmed cell death, such as thymocytes, dendritic cells, lymphocytes, macrophages and monocytes. There is some expression of P2X₇ receptors in normal homeostasis, such as on erythrocytes.

Interestingly, a P2X₇ receptor containing one or more monomers having a cis isomerisation at Pro210 and which is devoid of ATP binding function has been found on cells that are understood to be unable to undergo programmed cell death, such as preneoplastic cells and neoplastic cells. This conformational isoform of the receptor has been referred to as a “non functional” or “dysfunctional” receptor.

Antibodies generated from immunisation with a peptide including Pro210 in cis bind to dysfunctional P2X₇ receptors. However, they do not bind to P2X₇ receptors capable of forming an apoptotic pore under normal physiological conditions in the presence ATP. Accordingly, these antibodies are useful for selectively detecting many forms of carcinoma and haematopoietic cancers and for the treatment of some of these conditions.

Due to the nature of the conformational epitope present on dysfunctional P2X₇ receptors, it is not a straightforward task to identify and generate proteins for binding to this form of the receptor with sufficient affinity. Higher affinity reagents are generally desirable in applications for the detection and treatment of cancer.

There is a need for improved reagents for binding to dysfunctional P2X₇ receptors.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to antigen binding proteins comprising an antigen binding domain that binds to a P2X₇ receptor that has an impaired response to ATP such that it is unable to form an apoptotic pore under physiological conditions (i.e., a dysfunctional P2X₇ receptor). Preferably, the antigen binding proteins of the invention do not bind to P2X₇ receptors that function normally in response to ATP.

The invention provides an antigen binding protein for binding to dysfunctional P2X₇ receptor, the antigen binding protein comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
   wherein the sequence of any of the framework regions or complementarity determining regions are as described herein.

The invention provides an antigen binding protein for binding to nfP2X₇ receptor, the antigen binding protein comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
   wherein the sequence of any of the complementarity determining regions have an amino acid sequence as described in Table 1 below. Preferably, the framework regions have an amino acid sequence also as described in Table below, including amino acid variation at particular residues which can be determined by aligning the various framework regions derived from each antibody. The invention also includes where CDR1, CDR2 and CDR3 are sequences from the variable heavy chain of an antibody (a VH), CDR1a, CDR2a and CDR3a are sequences from the variable light chain of an antibody (a VL), or where CDR1, CDR2 and CDR3 are sequences from a VL, CDR1a, CDR2a and CDR3a are sequences from a VH.

In any embodiment, an antigen binding protein described herein comprises:
FR1-CDR1-FR2-CDR2-FR3-CDR3-FR4-linker-FR1a-CDR1a-FR2a-CDR2a-FR3a-CDR3a-FR4a.

As defined herein, the linker may be a chemical, one or more amino acids, or a disulphide bond formed between two cysteine residues.

In certain preferred embodiments, the invention provides an antigen binding protein comprising, consisting essentially of or consisting of the amino acid sequence of (in order of N to C terminus or C to N terminus) SEQ ID NOs: 4 and 12.

In a particularly preferred embodiment, the antigen binding protein comprises, consists essentially of or consists of, in order N to C terminus, SEQ ID NO: 12 and SEQ ID NO: 4 (ie VL to VH). Optionally the antigen binding protein comprises SEQ ID NO: 12 (VL)-linker-SEQ ID NO: 4 (VH).

In any embodiment, the invention provides an antigen binding protein that binds to or specifically binds to nfP2X₇ receptor and wherein the antigen binding protein competitively inhibits the binding of an antibody comprising a VH comprising a sequence as set forth in SEQ ID NO: 4 and a VL comprising a sequence as set forth in SEQ ID NO: 12.

The present invention also provides an antigen binding protein comprising an antigen binding domain of an antibody, wherein the antigen binding domain binds to or specifically binds to nfP2X₇ receptor, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 1, 29, 36 or 43, a CDR2 comprising a sequence at least about 80%, at least 85%, at least 90%, at least 92%, at least 95%, at least 97%, at least 99% identical to a sequence set in SEQ ID NO: 2, 30, 37 or 44 and a CDR3 comprising a sequence at least about 80%, at least 85%, at least 90%, at least 92%, at least 95%, at least 97%, at least 99% identical to a sequence set forth in SEQ ID NO: 3, 31, 38 or 45;
(ii) a VH comprising a sequence at least about 95% or 96% or 97% or 98% or 99% identical to a sequence set forth in SEQ ID NO: 4;
(iii) a VL comprising a CDR1 comprising a sequence at least about 80%, at least 85%, at least 90%, at least 92%, at least 95%, at least 97%, at least 99% identical to a sequence set forth in SEQ ID NO: 9, 50, 57 or 64, a CDR2 comprising a sequence at least about 80%, at least 85%, at least 90%, at least 92%, at least 95%, at least 97%, at least 99% identical to a sequence set forth in SEQ ID NO: 10, 51, 58 or 65, and a CDR3 comprising a sequence at least about 80%, at least 85%, at least 90%, at least 92%, at least 95%, at least 97%, at least 99% identical to a sequence set forth in SEQ ID NO: 11, 52, 59 or 66;
(iv) a VL comprising a sequence at least about 95% identical to a sequence set forth in SEQ ID NO: 12;
(v) a VH comprising a CDR1 comprising a sequence set forth in SEQ ID NO: 1, 29, 36 or 43, a CDR2 comprising a sequence set forth between in SEQ ID NO: 2, 30, 37 or 44, and a CDR3 comprising a sequence set forth in SEQ ID NO: 3, 31, 38 or 45;
(vi) a VH comprising a sequence set forth in SEQ ID NO: 4;
(vii) a VL comprising a CDR1 comprising a sequence set SEQ ID NO: 9, 50, 57 or 64, a CDR2 comprising a sequence set forth in SEQ ID NO: 10, 51, 58 or 65 and a CDR3 comprising a sequence set forth in SEQ ID NO: 11, 52, 59 or 66;
(viii) a VL comprising a sequence set forth in SEQ ID NO: 12;
(ix) a VH comprising a CDR1 comprising a sequence set forth in SEQ ID NO: 1, 29, 36 or 43, a CDR2 comprising a sequence set forth between in SEQ ID NO: 2, 30, 37 or 44 and a CDR3 comprising a sequence set forth in SEQ ID NO: 3, 31, 38 or 45; and a VL comprising a CDR1 comprising a sequence set SEQ ID NO: 9, 50, 57 or 64, a CDR2 comprising a sequence set forth in SEQ ID NO: 10, 51, 58 or 65 and a CDR3 comprising a sequence set forth in SEQ ID NO: 11, 52 59 or 66; or
(x) a VH comprising a sequence set forth in SEQ ID NO: 4 and a VL comprising a sequence set forth in SEQ ID NO: 12.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 5, 32, 39, or 46, a FR2 comprising a sequence at least about 80%, at least 85%, at least 90%, at least 92%, at least 95%, at least 97%, at least 99% identical to a sequence set in SEQ ID NO: 6, 33, 40 or 47, a FR3 comprising a sequence at least about 80%, at least 85%, at least 90%, at least 92%, at least 95%, at least 97%, at least 99% identical to a sequence set forth in SEQ ID NO: 7, 34, 41 or 48, and a FR4 comprising a sequence at least about 80%, at least 85%, at least 90%, at least 92%, at least 95%, at least 97%, at least 99% identical to a sequence set forth in SEQ ID NO: 8, 35, 42 or 49;
(ii) a VL comprising a FR1 comprising a sequence at least about 80%, at least 85%, at least 90%, at least 92%, at least 95%, at least 97%, at least 99% identical to a sequence set forth in SEQ ID NO: 13, 53, 60 or 67, a FR2 comprising a sequence at least about 80%, at least 85%, at least 90%, at least 92%, at least 95%, at least 97%, at least 99% identical to a sequence set forth in SEQ ID NO: 14, 54, 61 or 68, a FR3 comprising a sequence at least about 80%, at least 85%, at least 90%, at least 92%, at least 95%, at least 97%, at least 99% identical to a sequence set forth in SEQ ID NO: 15, 55, 62 or 69, and a FR4 comprising a sequence at least about 80%, at least 85%, at least 90%, at least 92%, at least 95%, at least 97%, at least 99% identical to a sequence set forth in SEQ ID NO: 16, 56, 63 or 70;
(iii) a VH comprising a FR1 comprising a sequence set forth in SEQ ID NO: 5, 32, 39 or 46, a FR2 comprising a sequence set forth between in SEQ ID NO: 6, 33, 40 or 47, a FR3 comprising a sequence set forth in SEQ ID NO: 7, 34, 41 or 48, and a FR4 comprising a sequence set forth in SEQ ID NO: 8, 35, 42 or 49;
(iv) a VL comprising a FR1 comprising a sequence set forth in SEQ ID NO: 13, 53, 60 or 67, a FR2 comprising a sequence set forth between in SEQ ID NO: 14, 54, 61 or 68, a FR3 comprising a sequence set forth in SEQ ID NO: 15, 55, 62 or 69, and a FR4 comprising a sequence set forth in SEQ ID NO: 16, 56, 63 or 70; or
(v) a VH comprising a FR1 comprising a sequence set forth in SEQ ID NO: 5, 32, 39 or 46, a FR2 comprising a sequence set forth between in SEQ ID NO: 6, 33, 40 or 47, a FR3 comprising a sequence set forth in SEQ ID NO: 7, 34, 41 or 48, and a FR4 comprising a sequence set forth in SEQ ID NO: 8, 35, 42 or 49; and a VL comprising a FR1 comprising a sequence set forth in SEQ ID NO: 13, 53, 60 or 67, a FR2 comprising a sequence set forth between in SEQ ID NO: 14, 54, 61 or 68, a FR3 comprising a sequence set forth in SEQ ID NO: 15, 55, 62 or 69, and a FR4 comprising a sequence set forth in SEQ ID NO: 16, 56, 63 or 70.

As described herein, the antigen binding protein may be in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody (including a BITE); or
(viii) one of (i) to (vii) linked to a constant region of an antibody, Fc or a heavy chain constant domain (CH) 2 and/or CH3.

In particularly preferred embodiments, the antigen binding protein of the present invention is a protein that does not comprise a constant region from an immunoglobulin. For example, preferably the antigen binding protein is an scFv, a dimeric scFv, an Fv fragment, a single domain antibody (dAb), a diabody, or fusion protein or conjugate comprising the same.

The foregoing antigen binding proteins can also be referred to as antigen binding domains of antibodies.

In certain embodiments, the complementarity determining region sequences (CDRs) of an antigen binding protein of the invention may be defined according to the IMGT numbering system, Kabat or Chothia systems.

Reference herein to a protein or antibody that “binds to” dysfunctional P2X₇ receptor (nfP2X₇ receptor) provides literal support for a protein or antibody that “binds specifically to” or “specifically binds to” nfP2X₇ receptor.

Preferably, an antigen binding protein as described herein is an antibody or antigen binding fragment thereof. Typically, the antigen binding protein is an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein.

As used herein the antigen binding protein may be a variable domain.

The present invention also provides an anti-nfP2X₇ receptor antibody or antigen binding fragment thereof, comprising a light chain variable region and a heavy chain variable region,

wherein said heavy chain variable region comprises:
a CDR H1 as set forth in SEQ ID NO: 1, 29, 36 or 43, a CDR H2 as set forth in SEQ ID NO: 2, 30, 37 or 44, and a CDR H3 as set forth in SEQ ID NO: 3, 31, 38 or 45; and
   wherein said light chain variable region comprises:
a CDR L1 as set forth in SEQ ID NO: 9, 50, 57 or 64, a CDR L2 as set forth in SEQ ID NO: 10, 51, 58 or 65 and a CDR L3 as set forth in SEQ ID NO: 11, 52, 59 or 66.

In any embodiment, the anti-nfP2X₇ receptor antibody or antigen binding fragment thereof, comprises a light chain variable region that comprises the sequence of SEQ ID NO: 12.

In any embodiment, the anti-nfP2X₇ receptor antibody or antigen binding fragment thereof, comprises a heavy chain variable region that comprises the sequence of SEQ ID NO: 4.

In any embodiment, the anti-nfP2X₇ receptor antibody or antigen binding fragment thereof, comprises a light chain variable region that comprises the sequence of SEQ ID NO: 12 and a heavy chain variable region that comprises the sequence of SEQ ID NO: 4.

In any embodiment, the anti-nfP2X₇ antibody or antigen binding fragment thereof comprises a light chain variable region that comprises a FR L1 as set forth in SEQ ID NO: 13, 53, 60 or 67, an FR L2 as set forth in SEQ ID NO: 14, 54, 61 or 68, a FR L3 as set forth in SEQ ID NO: 15, 55, 62 or 69 and a FR L4 as set forth in SEQ ID NO: 16, 56, 63 or 70.

In any embodiment of the invention, the anti-nfP2X₇ antibody or antigen binding fragment thereof comprises a heavy chain variable region that comprises a FR H1 as set forth in SEQ ID NO: 5, 32, 39 or 46, FR H2 as set forth in SEQ ID NO: 6, 33, 40 or 47, a FR H3 as set forth in SEQ ID NO: 7, 34, 41 or 48 and a FR H4 as set forth in SEQ ID NO: 8, 35, 42 or 49.

In any aspect of the invention and in any antigen binding protein described herein, there further includes an Fc region that is engineered to have reduced capacity to induce antibody-dependent cell-mediated cytotoxicity (ADCC). Preferably, the reduced capacity to induce ADCC is conferred by mutation, deletion or modification of amino acids in the Fc region which interact with an Fc receptor.

The invention provides an antigen binding protein as described herein wherein an amino acid sequence forming one or more of FR1, CDR1, FR2, CDR2, FR3, CDR3 and FR4 is a human sequence.

The invention provides an anti-nfP2X₇ antigen binding protein, immunoglobulin variable domain, antibody, dab, scFv, Fab, Fab′, F(ab′)2, Fv fragment, diabody, triabody, linear antibody, single-chain antibody molecule, or multispecific antibody comprising an antigen binding protein having a sequence as described herein, or including a CDR and/or FR sequence as described herein.

An antigen binding protein as described herein may comprise a human constant region, e.g., an IgG constant region, such as an IgG₁, IgG₂, IgG₃ or IgG₄ constant region or mixtures thereof. In the case of an antibody or protein comprising a V_{H} and a V_{L}, the V_{H} can be linked to a heavy chain constant region and the V_{L} can be linked to a light chain constant region.

In one example, an antigen binding protein as described herein comprises a constant region of an IgG₄ antibody or a stabilised constant region of an IgG₄ antibody. In one example, the protein or antibody comprises an IgG₄ constant region with a proline at position 241 (according to the numbering system of Kabat (Kabat et al., Sequences of Proteins of Immunological Interest Washington DC United States Department of Health and Human Services, 1987 and/or 1991)).

In one example, an antigen binding protein as described herein or a composition of an antigen binding protein as described herein, comprises a heavy chain constant region, comprising a stabilised heavy chain constant region, comprising a mixture of sequences fully or partially with or without the C-terminal lysine residue.

In one example, an antigen binding protein comprises a VH disclosed herein linked or fused to an IgG₄ constant region or stabilised IgG₄ constant region (e.g., as discussed above) and the VL is linked to or fused to a kappa light chain constant region.

In any aspect of the present invention, the antibody is a naked antibody. Specifically, the antibody is in a non-conjugated form and is not adapted to form a conjugate.

The invention also provides a conjugate in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody or fusion protein as described herein conjugated to a label or a cytotoxic agent.

In aspects of the invention directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
   wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
   wherein the first and second polypeptides associate to form an antigen binding protein of the present invention.

Examples of cells of the present invention include bacterial cells, yeast cells, insect cells or mammalian cells.

The invention provides a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides a vector comprising a nucleic acid described herein.

The invention provides a cell comprising a vector or nucleic acid described herein.

In any embodiment, the nucleic acid may comprise a nucleotide sequence as set forth in any of SEQ ID NOs: 71 to 86, preferably SEQ ID NO: 74 and/or 82, or sequences at least 80% identical thereto.

The invention provides a pharmaceutical composition comprising an antigen binding protein, or including a CDR and/or FR sequence as described herein, or an immunoglobulin variable domain, antibody, dab, scFv, Fab, Fab′, F(ab′)2, Fv fragment, diabody, triabody, linear antibody, single-chain antibody molecule, or multispecific antibody, fusion protein, or conjugate as described herein and a pharmaceutically acceptable carrier, diluent or excipient.

The invention provides a diagnostic composition comprising an antigen binding protein, or including a CDR and/or FR sequence as described herein, or antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein, a diluent and optionally a label.

The invention provides a kit or article of manufacture comprising an antigen binding protein, or including a CDR and/or FR sequence as described herein or an immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides use of a sequence according to one or more of CDR1, CDR2, FR1, FR2, FR3 and FR4 as described herein to produce an antigen binding protein for binding to a nfP2X₇ receptor.

The invention provides use of an antigen binding protein or a CDR and/or FR sequence as described herein to produce an anti nfP2X₇ receptor antigen binding protein having increased affinity for nfP2X₇ receptor.

The invention provides a library of nucleic acid molecules produced from the mutation of an antigen binding protein or a CDR and/or FR sequence as described herein, wherein at least one nucleic acid molecule in said library encodes an antigen binding protein for binding to an nfP2X₇ receptor.

The invention provides a method for producing an antigen binding protein for binding to a nfP2X₇ receptor as described herein comprising expressing a nucleic acid as described herein in a cell or animal as described herein.

The functional characteristics of an antigen binding protein of the invention will be taken to apply mutatis mutandis to an antibody of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

The invention provides a method for the prevention or treatment a condition or disease associated with expression of nfP2X₇ in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for said condition or disease. The disease or condition associated with expression of nfP2X₇ is preferably a cancer.

In another aspect, the present invention also provides for a method of treating or preventing a cancer in a subject, the method comprising administering an antigen binding protein of the invention to the subject, thereby treating or preventing a cancer in the subject. As used herein, methods of treating cancer include methods of inhibiting, preventing or minimising spread or progression of a cancer, including inhibiting or preventing metastasis of cancer.

In another aspect, the present invention also provides for the use of an antigen binding protein of the invention, in the manufacture of a medicament for the treatment or prevention of cancer in a subject.

In another aspect, the invention provides for an antigen binding protein or a pharmaceutical composition comprising an antigen binding protein of the invention, for use in the treatment or prevention of cancer in a subject.

As used herein, except where the context requires otherwise, the term “comprise” and variations of the term, such as “comprising”, “comprises” and “comprised”, are not intended to exclude further additives, components, integers or steps. The terms “comprising” and “including” are used interchangeably.

Further aspects of the present invention and further embodiments of the aspects described in the preceding paragraphs will become apparent from the following description, given by way of example and with reference to the accompanying drawings.

### Sequence Information

TABLE 1
sequence information

| Identifier | Region | SEQ ID NO: | Amino acid Sequence |
|---|---|---|---|
| BIL03 | HCDR1 | 1 | NHDMG |
| | (Kabat) | | |
| | HCDR2 | 2 | AISGSGGSTYYANSVKG |
| | (Kabat) | | |
| | HCDR3 | 3 | PKPMDTEFDY |
| | (Kabat) | | |
| | VH | 4 | EVQLLESGGGLVQPGGSLRLSCAASGFTFRNHDMGWV |
| | | | RQAPGKGLEWVSAISGSGGSTYYANSVKGRFTISRDNSK |
| | | | NTLYLQMNSLRAEDTAVYYCAEPKPMDTEFDYRSPGTLV |
| | | | TVSS |
| | HFR1 | 5 | EVQLLESGGGLVQPGGSLRLSCAASGFTFR |
| | (Kabat) | | |
| | HFR2 | 6 | WVRQAPGKGLEWVS |
| | (Kabat) | | |
| | HFR3 | 7 | RFTISRDNSKNTLYLQMNSLRAEDTAVYYCAE |
| | (Kabat) | | |
| | HFR4 | 8 | RSPGTLVTVSS |
| | (Kabat) | | |
| WT B1 | LCDR1 | 9 | RASQYIYDYLN |
| | (Kabat) | | |
| | LCDR2 | 10 | AASYLQS |
| | (Kabat) | | |
| | LCDR3 | 11 | QQYHHPST |
| | (Kabat) | | |
| | VL | 12 | DIQMTQSPSSLSASVGDRVTITCRASQYIYDYLNWYQQK |
| | | | PGKAPKLLIYAASYLQSGVPSRFSGSGSGTDFTLTISSLQ |
| | | | PEDFATYYCQQYHHPSTFGQGTKVEIKR |
| | LFR1 | 13 | DIQMTQSPSSLSASVGDRVTITC |
| | (Kabat) | | |
| | LFR2 | 14 | WYQQKPGKAPKLLIY |
| | (Kabat) | | |
| | LFR3 | 15 | GVPSRFSGSGSGTDFTLTISSLQPEDFATYYC |
| | (Kabat) | | |
| | LFR4 | 16 | FGQGTKVEIKR |
| | (Kabat) | | |
| G B1 | LCDR1 | 21 | RASQSISSYLN |
| | LCDR2 | 22 | AASSLQS |
| | LCDR3 | 23 | QQFDYMPLT |
| | VL | 24 | DIQMTQSPSSLSASVGDRVTITCRASQSISSYLNWYQQK |
| | | | PGKAPKLLIYAASSLQSGVPSRFSGSGSGTDFTLTISSLQ |
| | | | PEDFATYYCQQFDYMPLTFGQGTKVEIKR |
| | LFR1 | 25 | DIQMTQSPSSLSASVGDRVTITC |
| | LFR2 | 26 | WYQQKPGKAPKLLIY |
| | LFR3 | 27 | GVPSRFSGSGSGTDFTLTISSLQPEDFATYYC |
| | LFR4 | 28 | FGQGTKVEIKR |
| BIL03 | HCDR1 | 29 | GFTFRNH |
| | (Chothia) | | |
| | HCDR2 | 30 | SGSGGS |
| | (Chothia) | | |
| | HCDR3 | 31 | PKPMDTEFDY |
| | (Chothia) | | |
| | HFR1 | 32 | EVQLLESGGGLVQPGGSLRLSCAAS |
| | (Chothia) | | |
| | HFR2 | 33 | DMGWVRQAPGKGLEWVSAI |
| | (Chothia) | | |
| | HFR3 | 34 | TYYANSVKGRFTISRDNSKNTLYLQMNSLRAEDTAVYYC |
| | (Chothia) | | AE |
| | HFR4 | 35 | RSPGTLVTVSS |
| | (Chothia) | | |
| | HCDR1 | 36 | GFTFRNHD |
| | (IMGT) | | |
| | HCDR2 | 37 | ISGSGGST |
| | (IMGT) | | |
| | HCDR3 | 38 | AEPKPMDTEFDY |
| | (IMGT) | | |
| | HFR1 | 39 | EVQLLESGGGLVQPGGSLRLSCAAS |
| | (IMGT) | | |
| | HFR2 | 40 | MGWVRQAPGKGLEWVSA |
| | (IMGT) | | |
| | HFR3 | 41 | YYANSVKGRFTISRDNSKNTLYLQMNSLRAEDTAVYYC |
| | (IMGT) | | |
| | HFR4 | 42 | RSPGTLVTVSS |
| | (IMGT) | | |
| | HCDR1 | 43 | NHDMG |
| | (Martin) | | |
| | HCDR2 | 44 | AISGSGGSTYYANSVKG |
| | (Martin) | | |
| | HCDR3 | 45 | PKPMDTEFDY |
| | (Martin) | | |
| | HFR1 | 46 | EVQLLESGGGLVQPGGSLRLSCAASGFTFR |
| | (Martin) | | |
| | HFR2 | 47 | WVRQAPGKGLEWVS |
| | (Martin) | | |
| | HFR3 | 48 | RFTISRDNSKNTLYLQMNSLRAEDTAVYYCAE |
| | (v) | | |
| | HFR4 | 49 | RSPGTLVTVSS |
| | (Martin) | | |
| WTB1 | LCDR1 | 50 | RASQYIYDYLN |
| | (Chothia) | | |
| | LCDR2 | 51 | AASYLQS |
| | (Chothia) | | |
| | LCDR3 | 52 | QQYHHPST |
| | (Chothia) | | |
| | LFR1 | 53 | DIQMTQSPSSLSASVGDRVTITC |
| | (Chothia) | | |
| | LFR2 | 54 | WYQQKPGKAPKLLIY |
| | (Chothia) | | |
| | LFR3 | 55 | GVPSRFSGSGSGTDFTLTISSLQPEDFATYYC |
| | (Chothia) | | |
| | LFR4 | 56 | FGQGTKVEIK |
| | (Chothia) | | |
| | LCDR1 | 57 | QYIYDY |
| | (IMGT) | | |
| | LCDR2 | 58 | AAS |
| | (IMGT) | | |
| | LCDR3 | 59 | QQYHHPST |
| | (IMGT) | | |
| | LFR1 | 60 | DIQMTQSPSSLSASVGDRVTITCRAS |
| | (IMGT) | | |
| | LFR2 | 61 | LNWYQQKPGKAPKLLIY |
| | (IMGT) | | |
| | LFR3 | 62 | YLQSGVPSRFSGSGSGTDFTLTISSLQPEDFATYYC |
| | (IMGT) | | |
| | LFR4 | 63 | FGQGTKVEIK |
| | (IMGT) | | |
| | LCDR1 | 64 | QYIYDY |
| | (Martin) | | |
| | LCDR2 | 65 | AAS |
| | (Martin) | | |
| | LCDR3 | 66 | QQYHHPST |
| | (Martin) | | |
| | LFR1 | 67 | DIQMTQSPSSLSASVGDRVTITCRAS |
| | (Martin) | | |
| | LFR2 | 68 | LNWYQQKPGKAPKLLIY |
| | (Martin) | | |
| | LFR3 | 69 | YLQSGVPSRFSGSGSGTDFTLTISSLQPEDFATYYC |
| | (Martin) | | |
| | LFR4 | 70 | FGQGTKVEIK |
| | (Martin) | | |
| Identifier | Region | SEQ ID NO: | Nucleic acid Sequence |
| BIL03 | HCDR1 | 71 | aaccatgatatgggc |
| | (Kabat) | | |
| | HCDR2 | 72 | gcgattagcggcagcggcggcagcacctattatgcgaacagcgtgaaaggc |
| | (Kabat) | | |
| | HCDR3 | 73 | ccgaaaccgatggataccgaatttgattat |
| | (Kabat) | | |
| | VH | 74 | gaagtgcagctgctggaaagcggcggcggcctggtgcagccgggggcagc |
| | | | ctgcgcctgagctgcgcggcgagcggctttacctttcgcaaccatgatatgggc |
| | | | tgggtgcgccaggcgccgggcaaaggcctggaatgggtgagcgcgattagcg |
| | | | gcagcggcggcagcacctattatgcgaacagcgtgaaaggccgctttaccatt |
| | | | agccgcgataacagcaaaaacaccctgtatctgcagatgaacagcctgcgcg |
| | | | cggaagataccgcggtgtattattgcgcggaaccgaaaccgatggataccgaa |
| | | | tttgattatcgcagcccgggcaccctggtgaccgtgagcagc |
| | HFR1 | 75 | gaagtgcagctgctggaaagcggcggcggcctggtgcagccgggcggcagc |
| | (Kabat) | | ctgcgcctgagctgcgcggcgagcggctttacctttcgc |
| | HFR2 | 76 | tgggtgcgccaggcgccgggcaaaggcctggaatgggtgagc |
| | (Kabat) | | |
| | HFR3 | 77 | cgctttaccattagccgcgataacagcaaaaacaccctgtatctgcagatgaac |
| | (Kabat) | | agcctgcgcgcggaagataccgcggtgtattattgcgcggaa |
| | HFR4 | 78 | cgcagcccgggcaccctggtgaccgtgagcagc |
| | (Kabat) | | |
| WTB1 | LCDR1 | 79 | cgcgcgagccagagcattagcagctatctgaac |
| | (Kabat) | | |
| | LCDR2 | 80 | gcggcgagcagcctgcagagc |
| | (Kabat) | | |
| | LCDR3 | 81 | cagcagtttgattatatgccgctgacc |
| | (Kabat) | | |
| | VL | 82 | gatattcagatgacccagagcccgagcagcctgagcgcgagcgtgggcgatc |
| | | | gcgtgaccattacctgccgcgcgagccagagcattagcagctatctgaactggt |
| | | | atcagcagaaaccgggcaaagcgccgaaactgctgatttatgcggcgagcag |
| | | | cctgcagagcggcgtgccgagccgctttagcggcagcggcagcggcaccgat |
| | | | tttaccctgaccattagcagcctgcagccggaagattttgcgacctattattgccag |
| | | | cagtttgattatatgccgctgacctttggccagggcaccaaagtggaaattaaac |
| | | | gc |
| | LFR1 | 83 | gatattcagatgacccagagcccgagcagcctgagcgcgagcgtgggcgatc |
| | (Kabat) | | gcgtgaccattacctgc |
| | LFR2 | 84 | tggtatcagcagaaaccgggcaaagcgccgaaactgctgatttat |
| | (Kabat) | | |
| | LFR3 | 85 | ggcgtgccgagccgctttagcggcagcggcagcggcaccgattttaccctgac |
| | (Kabat) | | cattagcagcctgcagccggaagattttgcgacctattattgc |
| | LFR4 | 86 | tttggccagggcaccaaagtggaaattaaacgc |
| | (Kabat) | | |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Various antigen binding proteins, that bind to dysfunctional P2X₇ receptor are known, including single domain heavy chain antibodies. While such antibodies are useful for binding to dysfunctional P2X₇ receptor, they are often difficult to express and even when successfully expressed, they typically aggregate, making these molecule difficult to utilise for clinical applications. One solution to this problem is to pair the single heavy chain with a light chain counterpart. However, it is challenging to identify a suitable light chain pair for a given heavy chain, without impacting on affinity of binding to the target antigen. The present inventors have identified a suitable heavy light chain combination which has particular utility in binding dysfunctional P2X₇ receptor antigens, and peptide antigen derived therefrom.

### General and Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all the steps, features, compositions and compounds referred to or indicated in this specification, individually or collectively, and any and all combinations or any two or more of said steps or features.

One skilled in the art will recognise many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

For purposes of interpreting this specification, the following definitions will apply and whenever appropriate, terms used in the singular will also include the plural and vice versa. In the event that any definition set forth conflicts with any document incorporated herein by reference, the definition set forth below shall prevail.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilised in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989, Martin (“enhanced Chothia”; Mol Immunol. (2008) 45:3832-9; and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 17:

MPACCSCSDVFQYETNKVTRIQSMNYGTIKWFFHVIIFSYVCFALVSDK
LYQRKEPVISSVHTKVKGIAEVKEEIVENGVKKLVHSVFDTADYTFPLQ
GNSFFVMTNFLKTEGQEQRLCPEYPTRRTLCSSDRGCKKGWMDPQSKGI
QTGRCVVYEGNQKTCEVSAWCPIEAVEEAPRPALLNSAENFTVLIKNNI
DFPGHNYTTRNILPGLNITCTFHKTQNPQCPIFRLGDIFRETGDNFSDV
AIQGGIMGIEIYWDCNLDRWFHHCRPKYSFRRLDDKTTNVSLYPGYNFR
YAKYYKENNVEKRTLIKVFGIRFDILVFGTGGKFDIIQLVVYIGSTLSY
FGLAAVFIDFLIDTYSSNCCRSHIYPWCKCCQPCVVNEYYYRKKCESIV
EPKPTLKYVSFVDESHIRMVNQQLLGRSLQDVKGQEVPRPAMDFTDLSR
LPLALHDTPPIPGQPEEIQLLRKEATPRSRDSPVWCQCGSCLPSQLPES
HRCLEELCCRKKPGACITTSELFRKLVLSRHVLQFLLLYQEPLLALDVD
STNSRLRHCAYRCYATWRFGSQDMADFAILPSCCRWRIRKEFPKSEGQY
SGFKSPY

“P2X₇ receptor” may be a functional or non-functional receptor as described below. “P2X₇ receptor” encompasses naturally occurring variants of P2X₇ receptor, e.g., wherein the P2X₇ monomers are splice variants, allelic variants and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 17. In certain embodiments the P2X₇ receptor may have an amino acid sequence that is modified, for example various of the amino acids in the sequence shown in SEQ ID NO: 17 may be substituted, deleted, or a residue may be inserted.

“Functional P2X₇ receptor” generally refers to a form of the P2X₇ receptor having a binding site or cleft for binding to ATP. When bound to ATP, the receptor forms a pore-like structure that enables the ingress of calcium ions into the cytosol, one consequence of that may be programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes.

“Dysfunctional P2X₇ receptor” (also called “non-functional” or (nf) P2X₇) is a P2X₇ receptor that has an impaired response to ATP such that it is unable to form an apoptotic pore under physiological conditions. A dysfunctional P2X₇ receptor or (nfP2X₇ receptor) generally refers to a form of a P2X₇ receptor in which one or more of the monomers has a cis isomerisation at Pro210 (according to SEQ ID NO: 17). The isomerisation may arise from any molecular event that leads to misfolding of the monomer, including for example, mutation of monomer primary sequence or abnormal post translational processing. One consequence of the isomerisation is that the receptor is unable to bind to ATP, or otherwise binds ATP with a lower affinity than observed between ATP and receptors which do not contain an isomerisation at Pro210. In the circumstances, the receptor is unable to form a pore and this limits the extent to which calcium ions may enter the cytosol. Dysfunctional P2X₇ receptors are expressed on a wide range of epithelial, mesenchymal, neural, germinal and haematopoietic cancers. As used herein, a dysfunctional P2X₇ is preferably understood to be a P2X₇ receptor that has an impaired response to ATP such that it is unable to form an apoptotic pore under normal physiological conditions. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

The terms “E200”, “E300” and “composite” epitopes, refer to specific epitopes present on dysfunctional P2X₇ receptors, one or more of which are bound by the antigen binding proteins of the invention. “E200 epitope” generally refers to an epitope having the sequence GHNYTTNILPGLNITC (SEQ ID NO: 18). “E300 epitope” generally refers to an epitope having the sequence KYYKENNVEKRTLIK (SEQ ID NO: 19). A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 20).

The term “anti-P2X₇ receptor antibody” or “an antibody that binds to P2X₇ receptor” refers to an antibody that is capable of binding P2X₇ receptor with sufficient affinity such that the antibody is useful as a diagnostic and/or therapeutic agent in targeting P2X₇ receptor, typically non-functional P2X₇ receptor or a cancer associated P2X₇ receptor. Preferably, the extent of binding of a P2X₇ receptor antibody to an unrelated protein is less than about 10% of the binding of the antibody to P2X₇ receptor as measured, e.g., by a radioimmunoassay (RIA), Enzyme-Linked Immunosorbent Assay (ELISA), Biacore or Flow Cytometry. In certain embodiments, an antibody that binds to P2X₇ receptor has a dissociation constant (Kd) of <1 μM, <100 nM, <10 nM, <1 nM, or <0.1 nM. An anti nfP2X₇ receptor antibody is generally one having some or all of these serological characteristics and that binds to dysfunctional P2X₇ receptors but not to functional P2X₇ receptors.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. The protein may include one or more non-natural amino acids.

The term “polypeptide” or “polypeptide chain” will be understood from the foregoing paragraph to mean a series of contiguous amino acids linked by peptide bonds.

As used herein, the term “antigen binding domain” and shall be taken to mean a region of an antibody that is capable of specifically binding to an antigen, i.e., a V_{H} or a V_{L} or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanised antibodies, human antibodies, CDR-grafted antibodies, primatised antibodies, de-immunised antibodies, synhumanised antibodies, half-antibodies, bispecific antibodies).

An antibody generally comprises constant domains, which can be arranged into a constant region or constant fragment or fragment crystallisable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (C_{L} which is ˜110 amino acids in length) is aligned with and disulfide bonded to the first constant domain of the heavy chain (C_{H1} which is 330 to 440 amino acids in length). The light chain variable region is aligned with the variable region of the heavy chain. The antibody heavy chain can comprise 2 or more additional C_{H} domains (such as, C_{H2}, C_{H3} and the like) and can comprise a hinge region between the C_{H1} and C_{H2} constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example the antibody heavy chain is missing a C-terminal lysine residue. In one example, the antibody is humanised, synhumanised, chimeric, CDR-grafted or deimmunised.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR₁, CDR₂, and CDR₃, and framework regions (FRs). For example, the variable region comprises three or four FRs (e.g., FR₁, FR₂, FR₃ and optionally FR₄) together with three CDRs. V_{H} refers to the variable region of the heavy chain. V_{L} refers to the variable region of the light chain.

As used herein, the term “complementarity determining regions” (syn. CDRs; i.e., CDR₁, CDR₂, and CDR₃) refers to the amino acid residues of an antibody variable region the presence of which are major contributors to specific antigen binding. Each variable region domain (VH or VL) typically has three CDRs identified as CDR₁, CDR₂ and CDR₃. The CDRs of VH are also referred to herein as CDR H₁, CDR H₂ and CDR H₃, respectively, wherein CDR H₁ corresponds to CDR 1 of V_{H}, CDR H₂ corresponds to CDR 2 of V_{H} and CDR H₃ corresponds to CDR 3 of V_{H}. Likewise, the CDRs of V_{L} are referred to herein as CDR L₁, CDR L₂ and CDR L₃, respectively, wherein CDR L₁ corresponds to CDR 1 of V_{L}, CDR L₂ corresponds to CDR 2 of VL and CDR L₃ corresponds to CDR 3 of V_{L}. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196:901-917, 1987; Chothia et al., Nature 342:877-883, 1989; and/or Al-Lazikani et al., J. Mol. Biol. 273:927-948, 1997; the numbering system of Honnegher and Plükthun J. Mol. Biol. 309:657-670, 2001; or the IMGT system discussed in Giudicelli et al., Nucleic Acids Res. 25:206-211 1997.

In one example, the CDRs are defined according to the Kabat numbering system. Optionally, heavy chain CDR₂ according to the Kabat numbering system does not comprise the five C-terminal amino acids listed herein or any one or more of those amino acids are substituted with another naturally-occurring amino acid. In this regard, Padlan et al., FASEB J., 9:133-139, 1995 established that the five C-terminal amino acids of heavy chain CDR₂ are not generally involved in antigen binding.

“Framework regions” (FRs) are those variable region residues other than the CDR residues. The FRs of VH are also referred to herein as FR H₁, FR H₂, FR H₃ and FR H₄, respectively, wherein FR H₁ corresponds to FR 1 of VH, FR H₂ corresponds to FR 2 of V_{H}, FR H₃ corresponds to FR 3 of V_{H} and FR H₄ corresponds to FR 4 of V_{H}. Likewise, the FRs of V_{L} are referred to herein as FR L₁, FR L₂, FR L₃ and FR L₄, respectively, wherein FR L₁ corresponds to FR 1 of V_{L}, FR L₂ corresponds to FR 2 of V_{L}, FR L₃ corresponds to FR 3 of V_{L} and FR La corresponds to FR 4 of V_{L}.

As used herein, the term “Fv” shall be taken to mean any protein, whether comprised of multiple polypeptides or a single polypeptide, in which a V_{L} and a V_{H} associate and form a complex having an antigen binding domain, i.e., capable of specifically binding to an antigen. The V_{H} and the V_{L} that form the antigen binding domain can be in a single polypeptide chain or in different polypeptide chains. Furthermore, an Fv of the invention (as well as any protein of the invention) may have multiple antigen binding domains that may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH₂ or CH₃ domain, e.g., a minibody.

A “Fab fragment” consists of a monovalent antigen-binding fragment of an immunoglobulin and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH₃ domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to dysfunctional P2X₇ receptor with materially greater affinity (e.g., 1.5 fold or 2 fold or 5 fold or 10 fold or 20 fold or 40 fold or 60 fold or 80 fold to 100 fold or 150 fold or 200 fold) than it does to other related molecules, such as other purinergic receptors and in particular, than it does to functional P2X₇ receptor. In example of the present invention, an antigen binding protein that “specifically binds” to dysfunctional P2X₇ receptor with an affinity at least 1.5 fold or 2 fold or greater (e.g., 5 fold or 10 fold or 20 fold r 50 fold or 100 fold or 200 fold) than it does to functional P2X₇ receptor. Generally, but not necessarily, reference to binding means specific binding, and each term shall be understood to provide explicit support for the other term.

As used herein, the term “does not detectably bind” shall be understood to mean that an antigen binding protein, e.g. an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilised and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore or Blitz) in which the antigen binding protein is immobilised and contacted with an antigen.

As used herein, the term “epitope” (syn. “antigenic determinant”) shall be understood to mean a region of dysfunctional P2X₇ receptor to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three-dimensional structural characteristics, and/or specific charge characteristics.

As used herein, the terms “preventing”, “prevent” or “prevention” include administering an antigen binding protein of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding protein described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

The terms “engineered cell” and “genetically modified cell” as used herein can be used interchangeably. The terms mean containing and/or expressing a foreign gene or nucleic acid sequence that in turn modifies the genotype or phenotype of the cell or its progeny.

### Antibodies

In one example, an antigen binding protein as described herein according to any example is an antibody.

Methods for generating antibodies are known in the art and/or described in Harlow and Lane (editors) Antibodies: A Laboratory Manual, Cold Spring Harbor Laboratory, (1988). Generally, in such methods dysfunctional P2X₇ receptor or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunised animal at various points following immunisation. One or more further immunisations may be given, if required to achieve a desired antibody titre. The process of boosting and titreing is repeated until a suitable titre is achieved. When a desired level of immunogenicity is obtained, the immunised animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunised with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunisation, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunised animal are then fused with cells of an immortal myeloma cell, generally derived from the same species as the animal that was immunised with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titre, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison WI 53713, USA) is used to produce cell lines secreting MAbs (e.g., as described in Largaespada et al, J. Immunol. Methods. 197:85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present invention may be a synthetic antibody. For example, the antibody is a chimeric antibody, a humanised antibody, a human antibody synhumanised antibody, primatised antibody or a de-immunised antibody.

### Antibody Binding Domain-Containing Proteins

### Single Domain Antibodies

In some examples, a protein of the invention is or comprises a single-domain antibody (which is used interchangeably with the term “domain antibody” or “sdAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., U.S. Pat. No. 6,248,516).

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the VH of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fc (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly4Ser)3 being one of the more favoured linkers for a scFv.

The present invention also contemplates a disulfide stabilised Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Proteins Comprising Antigen Binding Domains Thereof

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980;
(iv) Fab₃ (e.g., as described in EP19930302894).

In any of the aforementioned antibody architectures, the binding domains of the proteins may be joined via a linker. For example, in the context of an scFv, the linker between the VH and VL may be a combination of one or more amino acid residues so as to provide a flexible linker. The skilled person will be familiar with suitable linker sequences to utilise. In typical examples, the linker is comprised of one or more glycine residues and serine residues. In one example, the linker may comprise the sequence G₄S (i.e., GGGGS) and the like. The linker may also comprise repeats of glycine and serine residues, such as (G₄S)₃, although it will be appreciated that any variations thereon may also be suitable, such as (G₄S)₃T.

### Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a V_{H} or V_{L} as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridises under stringent hybridisation conditions to a nucleic acid encoding an antigen binding site described herein. A “moderate stringency” is defined herein as being a hybridisation and/or washing carried out in 2×SSC buffer, 0.1% (w/v) SDS at a temperature in the range 45° C. to 65° C., or equivalent conditions. A “high stringency” is defined herein as being a hybridisation and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridisation solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157:105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525:309-322, 2009) or RNA (Kopsidas et al., Immunol. Lett. 107:163-168, 2006; Kopsidas et al. BMC Biotechnology, 7:18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370:389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG₁, IgG₂, IgG₃ and IgG₄. In one example, the constant region is human isotype IgG₄ or a stabilised IgG₄ constant region.

In one example, the Fc region of the constant region has a reduced ability to induce effector function, e.g., compared to a native or wild-type human IgG₁ or IgG₃ Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG₄ Fc region (i.e., from an IgG4 constant region), e.g., a human IgG₄ Fc region. Sequences of suitable IgG₄ Fc regions will be apparent to the skilled person and/or available in publicly available databases (e.g., available from National Center for Biotechnology Information).

In one example, the constant region is a stabilised IgG₄ constant region. The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG₄ antibody dissociates to form two molecules each containing a single heavy chain and a single light chain.

In one example, a stabilised IgG₄ constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG₄, this residue is generally a serine. Following substitution of the serine for proline, the IgG₄ hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues that are involved in inter-heavy chain disulfide bonds. It is generally defined as stretching from Glu226 to Pro243 of human IgG₁ according to the numbering system of Kabat. Hinge regions of other IgG isotypes may be aligned with the IgG₁ sequence by placing the first and last cysteine residues forming inter-heavy chain disulphide (S—S) bonds in the same positions (see for example WO2010/080538).

Additional examples of stabilised IgG₄ antibodies are antibodies in which arginine at position 409 in a heavy chain constant region of human IgG₄ (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG₁ Fc region comprising a substitution at one or more positions selected from the group consisting of 268, 309, 330 and 331. In another example, the Fc region is an IgG₁ Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276 (9): 6591-604, 2001). Additional examples of non-immunostimulatory Fc regions are described, for example, in Dall'Acqua et al., J Immunol. 177:1129-1138 2006; and/or Hezareh J Virol; 75:12161-12168, 2001).

In another example, the Fc region is a chimeric Fc region, e.g., comprising at least one CH₂ domain from an IgG₄ antibody and at least one CH₃ domain from an IgG₁ antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

### Protein Production

In one example, an antigen binding protein described herein according to any example is produced by culturing a hybridoma under conditions sufficient to produce the protein, e.g., as described herein and/or as is known in the art.

### Recombinant Expression

In another example an antigen binding protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilised on a solid or semi-solid support, washing the sample to remove unbound protein, and subsequently eluting the bound protein. Alternatively, or in addition a ligand or antibody that binds to a tag is used in an affinity purification method.

### Assaying Binding of Antigen Binding Proteins

It will be apparent to the skilled artisan that antigen binding protein of the present invention bind to dysfunctional P2X₇ receptor. Methods for assessing binding to a protein are known in the art, e.g., as described in Scopes (In: Protein purification: principles and practice, Third Edition, Springer Verlag, 1994). Such a method generally involves immobilising the antigen binding protein and contacting it with labelled antigen (dysfunctional P2X₇ receptor). Following washing to remove non-specific bound protein, the amount of label and, as a consequence, bound antigen is detected. Of course, the antigen binding protein can be labelled and the antigen immobilised. Panning-type assays can also be used. Alternatively, or additionally, surface plasmon resonance assays can be used.

Optionally, the dissociation constant (Kd), association constant (Ka) and/or affinity constant (KD) of an immobilised antigen binding protein for dysfunctional P2X₇ receptor or an epitope thereof is determined. The “Kd” or “Ka” or “KD” for a dysfunctional P2X₇ receptor-binding protein is in one example measured by a radiolabelled or fluorescently-labelled dysfunctional P2X₇ receptor ligand binding assay. In the case of a “Kd”, this assay equilibrates the antigen binding protein with a minimal concentration of labelled dysfunctional P2X₇ receptor or epitope thereof in the presence of a titration series of unlabelled dysfunctional P2X₇ receptor. Following washing to remove unbound dysfunctional P2X₇ receptor or epitope thereof, the amount of label is determined, which is indicative of the Kd of the protein.

According to another example the Kₐ, Kₐ or K_{D} is measured by using surface plasmon resonance assays, e.g., using BIAcore surface plasmon resonance (BIAcore, Inc., Piscataway, NJ) with immobilised dysfunctional P2X₇ receptor or a region thereof or immobilised antigen binding protein.

In some embodiments, the function of P2X₇ receptor may be decreased by at least 1%, 5%, 10%, 20%, 30%, 40%, 50%, 60%, 70%, 80%, 90% or greater than 99% upon binding by an antigen-binding protein, modified receptor or immune comprising same, of the present invention.

### Conditions to be Treated

The antigen binding proteins of the invention have particularly utility in the manufacture of medicaments (e.g., antibodies, antibody-drug conjugates), for the treatment of cancers that have dysfunctional P2X₇ receptor on their cell surface.

Examples of cancers which may be treated according to the methods of the present invention include, pre-neoplastic and neoplastic diseases. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukaemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukaemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukaemia (e.g. b-cell, mixed cell, null-cell, t-cell, t-cell chronic, htlv-ii-associated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

Other diseases and conditions include various inflammatory conditions. Examples may include a proliferative component. Particular examples include acne, angina, arthritis, aspiration pneumonia, disease, empyema, gastroenteritis, inflammation, intestinal flu, nee, necrotising enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

### Compositions

In some examples, an antigen binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers and preservatives.

The antigen binding proteins of the present invention may be formulated for local or topical administration, such as for topical application to the skin or tissue requiring treatment. Formulations for topical administration typically comprise a topical vehicle combined with active agent(s), with or without additional optional components. The pharmaceutical compositions of the invention may be in the form of a spray, cream, gel, lotion or the like for topical administration.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilising agents, suspending agents, emulsifying agents, viscosity adjusters, gelling agents, preservatives, antioxidants, skin penetration enhancers, moisturisers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturisers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturisers, emollients, fragrances, dyes/colorants, preservatives and other active ingredients that increase or enhance the efficacy of the final product.

Gels can be prepared with a range of viscosities, from thick or high viscosity to thin or low viscosity. These formulations, like those of lotions and creams, may also contain solvents, emulsifiers, moisturisers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturisers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminium silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilisers and antioxidants such as vitamin E, sodium ascorbate/ascorbic acid and propyl gallate. Suitable moisturisers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

Upon formulation, an antigen binding protein of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding protein of the present invention.

WO2002/080967 describes compositions and methods for administering aerosolized compositions comprising antibodies for the treatment of, e.g., asthma, which are also suitable for administration of an antigen binding protein of the present invention.

### Dosage and Timing of Administration

Suitable dosages of an antigen binding protein of the present invention will vary depending on the specific an antigen binding protein, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding protein(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding protein(s), rather the present invention encompasses any amount of the antigen binding protein(s) sufficient to achieve the stated result in a subject.

### Kits

The present invention additionally comprises a kit comprising one or more of the following:
(i) an antigen binding protein of the invention or expression construct(s) encoding same;
(ii) a cell of the invention;
(iii) a complex of the invention; or
(iii) a pharmaceutical composition of the invention.

In the case of a kit for detecting dysfunctional P2X₇ receptor, the kit can additionally comprise a detection means, e.g., linked to an antigen binding protein of the invention.

In the case of a kit for therapeutic/prophylactic use, the kit can additionally comprise a pharmaceutically acceptable carrier.

Optionally a kit of the invention is packaged with instructions for use in a method described herein according to any example.

### EXAMPLES

### Example 1: Identification of Light Chain Pairing for BIL03 Heavy Chain

Single chain human variable heavy regions are often poorly expressed and aggregate following expression, making them difficult to work with and to produce in clinically relevant amounts.

The inventors sought to identify a suitable light chain pairing for the single domain heavy chain protein BIL03 (as defined herein in Table 1).

More than 10 different light chain pairings were tested, including the variable light chain defined as GB1 in Table 1 herein.

The inventors found that the specific light chain pairing with WT B1 (defined in Table 1), provided for stable expression and reduced aggregation. Surprisingly, this light-heavy chain pairing also significantly improved binding affinity to target antigen, as demonstrated in the below table.

TABLE 2
binding affinity of BIL03 heavy chain compared to when
paired with various light chain variable domains

| Description | KD (M) | ka | kd | affinity (nM) (E200) |
|---|---|---|---|---|
| BIL03 VH only | 1.27E−07 | 2.0E+05 | 2.5E−02 | 127 |
| BIL03-WT B1 | 2.42E−08 | 5.41E+04 | 1.31E−03 | 24 |
| (scFv format) | | | | |
| BIL03- | 8.27E−08 | 3.14E+04 | 2.60E−03 | 83 |
| Germline B1 | | | | |
| BIL03-WT F5 | 2.03E−07 | 7.06E+04 | 1.43E−02 | 203 |
| BIL03-WT B4 | 4.22E−07 | 3.33E+04 | 1.41E−02 | 422 |
| BIL03- | 6.36E−07 | 1.88E+04 | 1.19E−02 | 636 |
| Germline G3 | | | | |

Binding affinity was assessed in relation to two different antigens derived from dysfunctional P2X₇ receptor: E200 peptide (GHNYTTRNILPGLNITC) and Ext peptide 17 (GHNYTTRNILPGLNITSTFHKTSGSGK).

It will be understood that the invention disclosed and defined in this specification extends to all alternative combinations of two or more of the individual features mentioned or evident from the text or drawings. All of these different combinations constitute various alternative aspects of the invention.

1. An antigen binding protein for binding to dysfunctional P2X₇ receptor, the antigen binding protein comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

2. The antigen binding protein of claim 1, wherein any one of the complementarity determining regions has an amino acid sequence as set forth in Table 1.

3. The antigen binding protein of claim 1 or 2, wherein the antigen binding protein competitively inhibits the binding of an antibody comprising a variable heavy chain (VH) comprising a sequence as set forth in SEQ ID NO: 4 and a variable light chain (VL) comprising a sequence as set forth in SEQ ID NO: 12.

4. The antigen binding protein of any one of claims 1 to 3, wherein CDR1, CDR2 and CDR3 are sequences from the variable heavy chain of an antibody (a VH), CDR1a, CDR2a and CDR3a are sequences from the variable light chain of an antibody (a VL), or where CDR1, CDR2 and CDR3 are sequences from the VL, CDR1a, CDR2a and CDR3a are sequences from VH.

5. The antigen binding protein of any one of claims 1 to 4, wherein the antigen binding protein described comprises:
FR1-CDR1-FR2-CDR2-FR3-CDR3-FR4-linker-FR1a-CDR1a-FR2a-CDR2a-FR3a-CDR3a-FR4a.

6. The antigen binding protein of claim 5, wherein the linker is a chemical, one or more amino acids, or a disulphide bond formed between two cysteine residues.

7. The antigen binding protein of any one of claims 1 to 6, wherein the antigen binding protein comprises, consists essentially of or consists of the amino acid sequence of (in order of N to C terminus or C to N terminus) SEQ ID NOs: 4 and 12, preferably wherein the antigen binding protein comprises the sequence, N to C terminus, SEQ ID NO: 12 and SEQ ID NO: 4.

8. The antigen binding protein of any one of claims 1 to 6, wherein the protein comprises an antigen binding domain that binds to or specifically binds to nfP2X₇ receptor, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 1, 29, 36 or 43, a CDR2 comprising a sequence at least about 80%, at least 85%, at least 90%, at least 92%, at least 95%, at least 97%, at least 99% identical to a sequence set in SEQ ID NO: 2, 30, 37 or 44 and a CDR3 comprising a sequence at least about 80%, at least 85%, at least 90%, at least 92%, at least 95%, at least 97%, at least 99% identical to a sequence set forth in SEQ ID NO: 3, 31, 38 or 45;
(ii) a VH comprising a sequence at least about 95% or 96% or 97% or 98% or 99% identical to a sequence set forth in SEQ ID NO: 4;
(iii) a VL comprising a CDR1 comprising a sequence at least about 80%, at least 85%, at least 90%, at least 92%, at least 95%, at least 97%, at least 99% identical to a sequence set forth in SEQ ID NO: 9, 50, 57 or 64, a CDR2 comprising a sequence at least about 80%, at least 85%, at least 90%, at least 92%, at least 95%, at least 97%, at least 99% identical to a sequence set forth in SEQ ID NO: 10, 51, 58 or 65, and a CDR3 comprising a sequence at least about 80%, at least 85%, at least 90%, at least 92%, at least 95%, at least 97%, at least 99% identical to a sequence set forth in SEQ ID NO: 11, 52, 59 or 66;
(iv) a VL comprising a sequence at least about 95% identical to a sequence set forth in SEQ ID NO: 12;
(v) a VH comprising a CDR1 comprising a sequence set forth in SEQ ID NO: 1, 29, 36 or 43, a CDR2 comprising a sequence set forth between in SEQ ID NO: 2, 30, 37 or 44, and a CDR3 comprising a sequence set forth in SEQ ID NO: 3, 31, 38 or 45;
(vi) a VH comprising a sequence set forth in SEQ ID NO: 4;
(vii) a VL comprising a CDR1 comprising a sequence set SEQ ID NO: 9, 50, 57 or 64, a CDR2 comprising a sequence set forth in SEQ ID NO: 10, 51, 58 or 65 and a CDR3 comprising a sequence set forth in SEQ ID NO: 11, 52, 59 or 66;
(viii) a VL comprising a sequence set forth in SEQ ID NO: 12;
(ix) a VH comprising a CDR1 comprising a sequence set forth in SEQ ID NO: 1, 29, 36 or 43, a CDR2 comprising a sequence set forth between in SEQ ID NO: 2, 30, 37 or 44 and a CDR3 comprising a sequence set forth in SEQ ID NO: 3, 31, 38 or 45; and a VL comprising a CDR1 comprising a sequence set SEQ ID NO: 9, 50, 57 or 64, a CDR2 comprising a sequence set forth in SEQ ID NO: 10, 51, 58 or 65 and a CDR3 comprising a sequence set forth in SEQ ID NO: 11, 52 59 or 66; or
(x) a VH comprising a sequence set forth in SEQ ID NO: 4 and a VL comprising a sequence set forth in SEQ ID NO: 12.

9. The antigen binding protein of claim 8, wherein the antigen binding domain further comprises at least one of:
i) a VH comprising a framework region (FR) 1 comprising a sequence at least about 80%, at least 85%, at least 90%, at least 92%, at least 95%, at least 97%, at least 99% identical to a sequence set forth in SEQ ID NO: 5, 32, 39, or 46, a FR2 comprising a sequence at least about 80%, at least 85%, at least 90%, at least 92%, at least 95%, at least 97%, at least 99% identical to a sequence set in SEQ ID NO: 6, 33, 40 or 47, a FR3 comprising a sequence at least about 80%, at least 85%, at least 90%, at least 92%, at least 95%, at least 97%, at least 99% identical to a sequence set forth in SEQ ID NO: 7, 34, 41 or 48, and a FR4 comprising a sequence at least about 80%, at least 85%, at least 90%, at least 92%, at least 95%, at least 97%, at least 99% identical to a sequence set forth in SEQ ID NO: 8, 35, 42 or 49;
(ii) a VL comprising a FR1 comprising a sequence at least about 80%, at least 85%, at least 90%, at least 92%, at least 95%, at least 97%, at least 99% identical to a sequence set forth in SEQ ID NO: 13, 53, 60 or 67, a FR2 comprising a sequence at least about 80%, at least 85%, at least 90%, at least 92%, at least 95%, at least 97%, at least 99% identical to a sequence set forth in SEQ ID NO: 14, 54, 61 or 68, a FR3 comprising a sequence at least about 80%, at least 85%, at least 90%, at least 92%, at least 95%, at least 97%, at least 99% identical to a sequence set forth in SEQ ID NO: 15, 55, 62 or 69, and a FR4 comprising a sequence at least about 80%, at least 85%, at least 90%, at least 92%, at least 95%, at least 97%, at least 99% identical to a sequence set forth in SEQ ID NO: 16, 56, 63 or 70;
(iii) a VH comprising a FR1 comprising a sequence set forth in SEQ ID NO: 5, 32, 39 or 46, a FR2 comprising a sequence set forth between in SEQ ID NO: 6, 33, 40 or 47, a FR3 comprising a sequence set forth in SEQ ID NO: 7, 34, 41 or 48, and a FR4 comprising a sequence set forth in SEQ ID NO: 8, 35, 42 or 49;
(iv) a VL comprising a FR1 comprising a sequence set forth in SEQ ID NO: 13, 53, 60 or 67, a FR2 comprising a sequence set forth between in SEQ ID NO: 14, 54, 61 or 68, a FR3 comprising a sequence set forth in SEQ ID NO: 15, 55, 62 or 69, and a FR4 comprising a sequence set forth in SEQ ID NO: 16, 56, 63 or 70; or
(v) a VH comprising a FR1 comprising a sequence set forth in SEQ ID NO: 5, 32, 39 or 46, a FR2 comprising a sequence set forth between in SEQ ID NO: 6, 33, 40 or 47, a FR3 comprising a sequence set forth in SEQ ID NO: 7, 34, 41 or 48, and a FR4 comprising a sequence set forth in SEQ ID NO: 8, 35, 42 or 49; and a VL comprising a FR1 comprising a sequence set forth in SEQ ID NO: 13, 53, 60 or 67, a FR2 comprising a sequence set forth between in SEQ ID NO: 14, 54, 61 or 68, a FR3 comprising a sequence set forth in SEQ ID NO: 15, 55, 62 or 69, and a FR4 comprising a sequence set forth in SEQ ID NO: 16, 56, 63 or 70.

10. The antigen binding protein of any one of claims 1 to 9, wherein the antigen binding protein is in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

11. The antigen binding protein of any one of claims 1 to 10, wherein the protein is in the form of:
(i) a diabody;
(ii) a triabody;
(iii) a tetrabody;
(iv) a Fab;
(v) a Fab′
(v) a F(ab′)2;
(vi) an Fv or fragment thereof;
(vii) a bispecific antibody or other form of multispecific antibody (including a BiTE);
(viii) one of (i) to (vii) linked to a constant region of an antibody, Fc or a heavy chain constant domain (CH) 2 and/or CH3; or
(ix) a linear antibody.

12. The antigen binding protein of any one of claims 1 to 11, wherein the protein is a recombinant or modified antibody.

13. The antigen binding protein of any one of claims 1 to 12, wherein the protein is a naked antibody.

14. The antigen binding protein of any one of claims 1 to 13, wherein the protein is an antibody that includes an Fc region that is engineered to have reduced capacity to induce antibody-dependent cell-mediated cytotoxicity (ADCC), preferably, wherein the reduced capacity to induce ADCC is conferred by mutation, deletion or modification of amino acids in the Fc region which interact with an Fc receptor.

15. The antigen binding protein of any one of claims 1 to 14, wherein the amino acid sequence forming one or more of FR1, CDR1, FR2, CDR2, FR3, CDR3 and FR4 is a human sequence.

16. The antigen binding protein of any one of claims 1 to 15, wherein the protein comprises a human constant region of an immunoglobulin.

17. The antigen binding protein of any one of claims 1 to 16, wherein the protein comprises a heavy chain constant region, optionally comprising a stabilised heavy chain constant region, optionally comprising a mixture of sequences fully or partially with or without the C-terminal lysine residue.

18. A conjugate comprising an antigen binding protein of any one of claims 1 to 17, conjugated to a label including a radiolabel, a cytotoxic agent or other chemical active agent.

19. A fusion protein comprising an antigen binding protein of any one of claims 1 to 18.

20. A nucleic acid encoding an antigen binding protein, or fusion protein of any one of claims 1 to 19; optionally wherein the nucleic acid comprises a nucleotide sequence as set forth in any of SEQ ID NOs: 71 to 86, preferably SEQ ID NO: 74 or 82, or sequences at least 80% identical thereto.

21. An expression construct comprising a nucleic acid of claim 20.

22. A cell comprising a vector or nucleic acid of claim 20 or 21.

23. A pharmaceutical composition comprising an antigen binding protein of any one of claims 1 to 17, a conjugate of claim 18, a fusion protein of claim 19 or a cell of claim 22, and a pharmaceutically acceptable carrier, diluent or excipient.

24. A diagnostic composition comprising an antigen binding protein of any one of claims 1 to 17, a conjugate of claim 18, a fusion protein of claim 19 or a cell of claim 22, a diluent and optionally a label.

25. A kit or article of manufacture comprising an antigen binding protein of any one of claims 1 to 17, a conjugate of claim 18, a fusion protein of claim 19 or a cell of claim 22.

26. A method of treating or preventing a cancer in a subject, the method comprising administering an antigen binding protein of any one of claims 1 to 17, a conjugate of claim 18, a fusion protein of claim 19 or a cell of claim 22, to the subject, thereby treating or preventing a cancer in the subject.

27. Use of an antigen binding protein of any one of claims 1 to 17, a conjugate of claim 18, a fusion protein of claim 19 or a cell of claim 22, in the manufacture of a medicament for the treatment or prevention of cancer in a subject.

28. An antigen binding protein of any one of claims 1 to 17, a conjugate of claim 18, a fusion protein of claim 19 or a cell of claim 22, for use in the treatment or prevent of cancer.
